(19) United States
(12) Patent Application Publication
(10) Pub. No.: US 2017/0259241 A1
(43) Pub. Date: Sep. 14, 2017

(54) A MANUFACTURING PROCESS FOR AN IRON CONTAINING ADSORBENT

(71) Applicant: LUPIN LIMITED, Mumbai, Maharashtra (IN)

(72) Inventors: Gaurav KUMAR, Pune, Maharashtra (IN); Ashok Sopan YADAV, Pune, Maharashtra (IN); Nitesh Yashwant AMRUTKAR, Pune, Maharashtra (IN); Surinder Kumar ARORA, Pune, Maharashtra (IN); Radhakrishna SHIVDAVKAR, Pune, Maharashtra (IN); Purna Chandra RAY, Pune, Maharashtra (IN); Girij Pal SINGH, Pune, Maharashtra (IN)

(21) Appl. No.: 15/510,548

(22) PCT Filed: Sep. 9, 2015
(86) PCT No.: PCT/IB2015/056892

§ 371 Date: Mar. 10, 2017

(30) Foreign Application Priority Data
Sep. 10, 2014 (IN) 2888/MUM/2014
May 26, 2015 (IN) 2053/MUM/2015

(51) Int. Cl. B01J 20/06; B01J 20/24; B01J 20/28; A61K 33/26; B01J 20/04

(52) U.S. Cl. CPC B01J 20/06; A61K 33/26; B01J 20/043; B01J 20/28059; B01J 20/28004; B01J 20/24

(57) ABSTRACT

The present invention is directed towards a manufacturing method for an iron containing phosphate adsorbent. Said iron containing phosphate adsorbent is characterized by a PSD d(0.9) in the range of 20 μm to 80 μm, a BET active surface area less than 15 m²/gm and phosphate binding in the range of 35 to 60 mg/gm.

### FIELD OF THE INVENTION

The present invention relates to an industrially viable process for the manufacturing of an iron containing phosphate adsorbent, uses thereof and pharmaceutical compositions containing it.

### BACKGROUND OF THE INVENTION

Hyperphosphatemia is associated with significant increase in morbidity and mortality, and may induce severe complications, such as hypocalcemia, decreasing of vitamin-D production and metastatic calcification. Hyperphosphatemia is also contributing to the increased incidence of cardiovascular disease among dialysis-dependent patients.

The phosphate binding capacity of iron oxide hydroxides is known in the art. The possible medical application of iron hydroxides and iron oxide hydroxides as phosphate adsorbents is also described.

U.S. Pat. No. 4,970,079 patent discloses a method of controlling serum phosphate level in patients by iron oxy-hydroxides which bind to ingested phosphate. U.S. Pat. No. 5,514,281 patent also discloses a process for the selective elimination of inorganic phosphate from body fluids by using a polynuclear metal oxyhydroxide preferably iron (III) oxyhydroxide.

U.S. Pat. No. 6,174,442 patent describes an adsorbent for phosphate and a process for the preparation thereof, which contains polynuclear (3-iron hydroxide stabilized by carbohydrates and/or humic acid.

In order to obtain an iron-based compound which can be used as a pharmaceutical, it is necessary to have an iron-based compound which is stable. It is known that iron oxide-hydroxide is not a stable compound with time ageing occurs. Ageing usually not only involves crystallization but also particle enlargement. Such ageing may alter the phosphate binding of an iron oxide-hydroxide based phosphate adsorbent.

Accordingly, there exists a need for a process for manufacturing of an iron containing phosphate adsorbent. The process needs to be scalable, robust and consistently producing an iron containing phosphate adsorbent of the required pharmaceutical grade.

### OBJECT OF THE INVENTION

It is an object of the present invention to provide a process for the manufacture of an iron containing phosphate adsorbent, which is suitable for the preparation of a pharmaceutical composition for the treatment of hyperphosphatemia.

It is another object of the present invention to provide an industrial viable process for the manufacturing of an iron containing phosphate adsorbent.

It is yet another object of the present invention to provide an iron containing phosphate adsorbent, wherein the said iron containing phosphate adsorbent comprises particles having 90% distribution in the range of 20 μm to 80 μm.

It is yet another object of the present invention to provide an iron containing phosphate adsorbent, wherein the said iron containing phosphate adsorbent comprises particles having a BET surface area of 0.1 to 50 m²/gm; preferably, 0.5 to 25 m²/gm; more preferably, 1 to 15 m²/gm.

It is yet another object of the present invention to provide a pharmaceutical composition comprising an effective amount of an iron containing phosphate adsorbent which is obtained by following the process of the present invention.

### BRIEF DESCRIPTION OF ACCOMPANYING FIGURE

FIG. 1: PXRD pattern of the iron containing phosphate adsorbent according to the present invention.

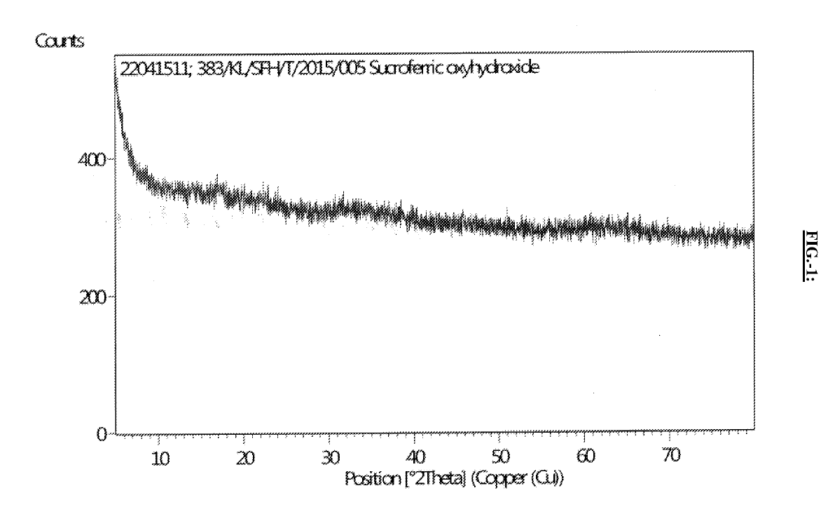

FIG. 2: Schematic diagram outlining the method of manufacturing the iron containing phosphate adsorbent according to the present invention.

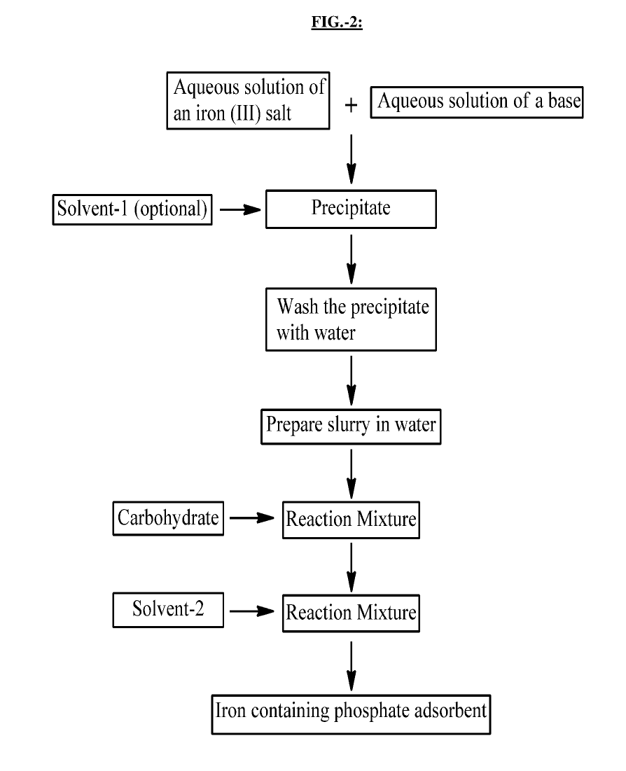

### DESCRIPTION OF THE INVENTION

The present invention relates to a process for the manufacture of an iron containing phosphate adsorbent, pharmaceutical compositions containing it and uses thereof.

The present invention further relates to an industrially viable process for the manufacturing of an iron containing phosphate adsorbent, pharmaceutical compositions containing it and uses thereof.

An embodiment of the invention provides a manufacturing and quality control process/analysis for making a pharmaceutical grade iron containing phosphate adsorbent.

The process of the present invention can be implemented at large scale to produce multi-kilogram batches of pharmaceutical grade iron containing phosphate adsorbent.

The present invention further relates to an iron containing phosphate adsorbent, wherein the said iron containing phosphate adsorbent comprises particles having a BET (Brunauer-Emmett-Teller) surface area of 0.1 to 50 m²/gm; preferably, 0.5 to 25 m²/gm; more preferably, 1 to 15 m²/gm.

The analysis of active surface area is based on BET theory which describes the phenomenon of mass and energy interaction and phase changes during gas adsorption onto solid surfaces and in pore spaces. In BET active surface area measurement, the volume of a monolayer of gas is determined which allows the surface area of the sample to be determined using the area occupied by a single layer of adsorbed gas molecule.

The present invention further relates to an iron containing phosphate adsorbent, having Particle Size Distribution (PSD) d(0.9) in the range of 20 μm to 80 μm.

As used herein, “Particle Size Distribution (PSD)” means the cumulative volume size distribution of equivalent spherical diameters as determined by laser diffraction in Malvern Master Sizer equipment or its equivalent.

The important characteristic of the PSD is d(0.9), which is the size, in microns, below which 90% of the particles by volume are found, Thus, for example, a d(0.9) of less than 80 microns means that 90 volume-percent of the particles in a composition have a diameter less than 80 microns.

Preferably, the iron containing phosphate adsorbent is sucroferric oxyhydroxide which is a mixture of polynuclear iron (III) oxyhydroxide or polynuclear iron (III) hydroxide stabilized by sucrose and starches.

As is known to a person skilled in the art, “phosphate adsorbents” are compounds or compositions that are capable to act as an adsorbent for phosphate from aqueous medium, for example from aqueous solutions, in particular from physiological aqueous solutions. They are particularly suitable as an adsorbent for inorganic phosphate and phosphate bonded to foodstuffs, especially in a preparation for oral application for the prophylaxis and treatment of hyperphosphatemia conditions, in particular in patients with chronic renal insufficiency, which have a pathologically increased serum phosphate level.

The phosphate adsorbent, e.g. sucroferric oxyhydroxide, may be combined with one or more pharmaceutically acceptable carriers and, optionally, one or more other conventional pharmaceutical adjuvants and administered orally, in the form of tablets, chewable tablets, mini-tablets (micro-tablets), granules, capsules, caplets, granules, powders etc. The compositions may be prepared by conventional means or enabling technologies.

The present invention relates to a process for the manufacturing of an iron containing phosphate adsorbent, which comprises the steps of:
(1) Mixing an aqueous solution of an iron (III) salt with an aqueous solution of a base optionally in the presence of solvent-1, to form a precipitate of ferric hydroxide;
(2) Isolating the precipitate and optionally washing with water;
(3) Slurrying the precipitate in water to obtain a suspension;
(4) Adding a carbohydrate and/or humic acid to the suspension;
(5) Adding solvent-2 to the suspension to obtain an iron containing phosphate adsorbent; and
(6) Isolating the iron containing phosphate adsorbent.

According to the present invention, the iron (III) salt comprises of iron (III) chloride, iron (III) nitrate or iron (III) sulfate; preferably the iron salt comprises of iron (III) chloride, e.g. solid iron (III) chloride hexahydrate.

As used herein, the term “iron (III) salt” also includes iron (II) salts. In a preferred aspect, the presence of iron (II) salt in iron (III) salt is up to 10% w/w; more preferably, up to 5% w/w.

According to the present invention, the aqueous solution of iron (III) salt may be in particular a solution of iron (III) salt, as herein above defined, in water.

According to the present invention, the base is a hydroxide or a carbonate of an alkali or alkaline earth metal. Alkali carbonates, alkali bicarbonates and alkali metal hydroxides (e.g. of sodium) are preferred as bases. In particular, the base is LiOH, KOH, NaOH, NaHCO₃ Na₂CO₃, Ca(OH)₂, Mg(OH)₂, Li₂CO₃, K₂CO₃, CaCO₃ or MgCO₃; preferably Na₂CO₃.

As used herein, the term “ferric hydroxide” includes a solid, a semi-solid or a suspension comprising of ferric hydroxide, ferric oxyhydroxide, ferric oxide hydroxide, ferric oxide or mixture thereof.

According to the present invention, the aqueous base may comprise of an aqueous solution containing a base as hereinabove defined, in water.

According to the present invention, the amount of the base is used in order to obtain the desired pH to produce the colloidal suspension or precipitate of ferric hydroxide, e.g. to adjust the pH of the solution resulting from the mixing the aqueous solution of the base to a pH between about 3 and about 10, preferably between about 6 and about 8, more preferably about 7.

According to the present invention, a water miscible solvent-1 may be optionally added in the step-1, such as straight or branched chain alcohols, ketones or ethers. Preferably, such an alcohol is methanol, ethanol, isopropanol, n-propanol or a combination thereof. In most preferred aspect, such alcohol is ethanol. Preferably, such a ketone is acetone. Preferably, such an ether is tetrahydrofuran, 1,4-dioxane or a combination thereof.

According to the present invention, the reaction, in particular the step (1), is preferably done at a temperature between about 1° C. and about 45° C., preferably between about 2° C. and about 25° C., more preferably between 5 to 15° C.

According to the present invention, in the step (2) the obtained ferric hydroxide precipitate is isolated by techniques known in the art, e.g. by siphoning, decantation, filtration, centrifugation, and then washed with water. The precipitate is washed once or several times, preferably three times with water.

According to the present invention, the product is then suspended in water. A minimum amount of water is needed so that the suspension is processed. For example the ratio amount of water/phosphate adsorbent is from about 0.4 to about 5, preferably 0.5 to 3, more preferably 0.5 to 2.

According to the present invention, the carbohydrate is a soluble and/or an insoluble carbohydrate.

According to the present invention, the soluble carbohydrate is a glucose derivative. Glucose derivatives are agarose, dextran, dextrin, dextran derivatives, cellulose, cellulose derivatives, sucrose, maltose, lactose, mannitol or mixture thereof. Preferred glucose derivatives are sucrose, maltodextrin or a mixture thereof. Most preferred glucose derivative is sucrose.

According to the present invention, the amount of soluble carbohydrate, e.g. glucose derivative, added in step (4) is about 5 to about 40 weight %, preferably about 10 to about 35 weight %, based on the weight of the phosphate adsorbent. Preferably about 10 to about 35 weight % sucrose or about 15 to about 30 weights % sucrose is used.

According to the present invention, the insoluble carbohydrate is starch. Starch is corn starch, wheat starch, rice starch, maize starch, pea starch, potato starch, pregelatinized maize starch or mixture thereof. Preferably starch is a mixture of potato starch and pregelatinized maize starch.

In the step (5), solvent-2 is added. In a preferred aspect of the present invention, the solvent is straight or branched chain alcohols, ketones, ethers or a mixture thereof. In another preferred aspect, such alcohol is methanol, ethanol, isopropanol, n-propanol or a mixture thereof. In another preferred aspect, such ketone is acetone. In another preferred aspect, such ether is tetrahydrofuran, 1,4-dioxane or a mixture thereof.

According to the present invention, the step (6) comprises of isolating the phosphate adsorbent. Such isolation is carried out using techniques known in the art. In a preferred aspect of the present invention, the isolation is carried out with filtration, centrifugation or spray drying technique.

Optionally, the product is dried under vacuum at about 20 to 50° C., preferably at about 25 to 35° C. for 5-24 hours, preferably for 5-18 hours, or more preferably for 10-12 hours.

According to the present invention, there is provided an industrially viable process for manufacturing an iron containing phosphate adsorbent having high phosphate binding capacity in form of a dry powder.

The iron containing phosphate adsorbent obtained by the process of the present invention has a BET surface area of 0.1 to 50 m²/gm; preferably, 0.5 to 25 m²/gm; more preferably, 1 to 15 m²/gm.

The iron containing phosphate adsorbent obtained by the process of the present invention has a Particle Size Distribution (PSD) d(0.9) in the range of 20 μm to 80 μm.

The iron containing phosphate adsorbent obtained by the process of the present invention has x-ray amorphous.

Furthermore a formulation step can be performed following step (6). For example mixing, granulating, encapsulating and/or tableting the phosphate adsorbent may be done, with adequate excipients if necessary.

According to the present invention, pharmaceutical composition of the invention refers to pharmaceutical composition containing the iron containing phosphate adsorbent.

The iron containing phosphate adsorbent according to the invention is, therefore, useful in the treatment and/or prevention of hyperphosphatemia, hypercalcemia, hyperparathyroidism reduction, in cardiovascular morbidity and mortality, renal osteodystrophy, calciphylaxis and soft tissue calcifications. In particular the iron containing phosphate adsorbent according to the present invention is suitable for the treatment and/or prevention of hyperphosphatemia in humans.

The phosphate adsorbent of the present invention and pharmaceutical composition containing it are more particularly useful in patients with hyperphosphatemia, e.g. for dialysis-dependent patients, e.g. hemodialysis, or patients suffering from advanced chronic kidney diseases (CKD), chronic renal failure, chronic renal insufficiency, end-stage renal disease.

In another embodiment of the invention, the phosphate adsorbent of the invention and pharmaceutical compositions containing it, are also useful for selectively removing inorganic phosphate or eliminating inorganic phosphate from dialysis fluids, whole blood or plasma; e.g. in patients on dialysis, e.g. on chronic hemodialysis, by administering to said subject an effective amount of the iron containing phosphate adsorbent according to the present invention.

Pharmaceutical compositions according to the present invention may be formulated in any conventional form, preferably or dosage forms, preferred formulations are powder, granulate, tablet, for example dispersible tablet. Such pharmaceutical composition can be prepared by the methods known in the literature.

### EXAMPLES

In examples which are intended to illustrate embodiments of the invention but which are not intended to limit the scope of the invention:

1) Method of Making an Iron Containing Phosphate Adsorbent

To a solution of 1.96 kg sodium carbonate dissolved in 12.5 liter water, solution of 2.5 kg iron (III) chloride hexahydrate dissolved in 17.5 liter water was added at a temperature of 5-10° C. The resulting mixture was stirred for 90 to 120 minutes at 5-10° C. (25.0×3) liter water was added to the reaction mass and raised the temperature at 15-20° C. with stirring. Stopped the stirring, settled precipitate and the supernatant water was removed. The precipitate was filtered and washed with 1.25 liter water. A suspension of the precipitate was prepared in water. To this, 875.0 gm sucrose and 695.0 gm potato starch were added and stirred for 120 minutes at 25-35° C. Cooled the reaction mass at 10-15° C. and stirred for 90 to 120 minutes. 25.0 liters cold acetone was added to the reaction mass at 10-15° C. and stirred for 90 to 120 minutes. The final product was filtered and washed with 1.25 liter cold acetone and further dried under vacuum at 30-35° C.

Yield: 2.08 kg

2) Large-Scale Method of Making an Iron Containing Phosphate Adsorbent

An aqueous solution of sodium carbonate and an aqueous solution of iron (III) chloride hexahydrate were mixed at a temperature of 5-10° C., optionally in the presence of solvent-1. A volume of aqueous solution of sodium carbonate necessary to maintain the pH at about 7.0 to form a colloidal suspension of ferric hydroxide. The resulting mixture was stirred for 90 to 120 minutes at 5-10° C. Water was added to the reaction mass with stirring. Stopped the stirring, settled precipitated product and the water was decanted or siphoned. The precipitated product was further filtered and washed with using water. Suspension of the precipitated product was prepared in the water. Subsequently, sucrose and starch were added in to the suspension and stirred for 120 minutes at 25-35° C. Cooled the reaction mixture at 10-15° C. and stirred for 90 to 120 minutes. Solvent-2 was added to the reaction mixture at 10-15° C. and stirred for 90 to 120 minutes. The product was filtered and washed with the solvent-2 and further dried under vacuum at 30-35° C.

Few illustrative examples provided in Table-1, wherein the iron containing phosphate adsorbents were prepared according to the process of example-2 using the respective combination of Solvent-1 and Solvent-2 as given in the table:

TABLE 1

| Example | | | PXRD of Iron Containing |
|---|---|---|---|
| No. | Solvent-1 | Solvent-2 | phosphate adsorbent |
| 2a | Ethanol | Acetone | Amorphous |
| 2b | Acetone | Ethanol | Amorphous |
| 2c | Acetone | Acetone | Amorphous |
| 2d | — | THF | Amorphous |
| 2e | — | Methanol | Amorphous |

3) Physical Properties of an Iron Containing Phosphate Adsorbents Prepared as Per Above Example-2.

BET Active Surface Area:
Instrument: Surface area analyzer
Condition: Surface area (m²/gm) at N2.P/P0=10%

TABLE 2

| | Sucrose | Starch | BET active |
|---|---|---|---|
| | content | content | Surface Area |
| Example no. | (% w/w) | (% w/w) | (m²/gm) |
| 3a | 28.30 | 28.18 | 7.6728 |
| 3b | 28.40 | 28.00 | 6.6759 |
| 3c | 29.20 | 25.25 | 3.8580 |

Phosphate Binding Capacity at pH 3.0:
Method: Ion Chromatography
Instrument: Metrohm IC equipped with pump, Injector, conductivity detector and recorder.
Column: Dionex Ion Pac AS-11 (4.0×250mm), 13 μm
Guard column: Dionex Ion Pac AG-11 (4.0×50mm), 13 μm
Buffer preparation: Weigh accurately about 2.118 g of Sodium carbonate and 180 mg of Sodium hydroxide in 1700 mL water.
Mobile phase preparation: Buffer and acetonitrile (1700:300).

Results: Phosphate binding of an iron containing phosphate adsorbents obtained by following the process of the present invention found in the range of 30 mg/gm to 60 mg/gm.

Particle Size Distribution:
Instrument Model: Malvern Mastersizer 2000 Particle size analyzer
Sampling Unit: Hydro 2000S
Analysis Model: General Purpose
Dispersant: 0.1% Span 85 in n-Hexane
Dispersant RI: 1.380
Stirrer Speed: 2200 RPM
Absorption: 1
Particle RI: 1.5
Obscuration: 10% to 20%
Sample Measurement time: 12 seconds
Background Measurement time: 12 seconds

TABLE 3

| | Particle size distribution |
|---|---|
| Example no. | d(0.9) (μm) |
| 3d | 43.67 |
| 3e | 65.37 |
| 3f | 37.75 |

1. A process for the preparation of an iron containing phosphate adsorbent, which process comprises the steps of:
(1) Mixing an aqueous solution of an iron (III) salt with an aqueous solution of a base optionally in the presence of solvent-1 to form a precipitate;
(2) Isolating the precipitate and optionally washing with water;
(3) Slurrying the precipitate in water to obtain a suspension;
(4) Adding a carbohydrate and/or humic acid to the suspension;
(5) Adding solvent-2 to the suspension to obtain an iron containing phosphate adsorbent; and
(6) Isolating the iron containing phosphate adsorbent.

2. The process for the preparation of an iron containing phosphate adsorbent of claim-1, wherein the iron (III) salt is iron (III) chloride, iron (III) nitrate or iron (III) sulfate.

3. The process for the preparation of an iron containing phosphate adsorbent of claim-2, wherein the iron (III) salt is an iron (III) chloride.

4. The process for the preparation of an iron containing phosphate adsorbent of claim-1, wherein the base is a hydroxide or a carbonate of alkali or alkaline earth metals.

5. The process for the preparation of an iron containing phosphate adsorbent of claim-4, wherein the base is selected from LiOH, KOH, NaOH, NaHCO₃ Na₂CO₃, Ca(OH)₂, Mg(OH)₂, Li₂CO₃, K₂CO₃, CaCO₃, MgCO₃ and a mixture thereof.

6. The process for the preparation of an iron containing phosphate adsorbent of claim-1, wherein the solvent-1 is selected from methanol, ethanol, isopropanol, n-propanol, butanol and a combination thereof.

7. The process for the preparation of an iron containing phosphate adsorbent of claim-1, wherein the carbohydrate is a soluble carbohydrate or an insoluble carbohydrate.

8. The process for the preparation of an iron containing phosphate adsorbent of claim-1, wherein the solvent-2 is selected from an alcohol, ketone, ether, ester and a mixture thereof.

9. An iron containing phosphate adsorbent having 90% of the particle by volume in the range of 20 μm to 80 μm.

10. An iron containing phosphate adsorbent having a BET active surface area less than 15 m²/gm.

11. A pharmaceutical composition comprising an iron containing phosphate adsorbent according to claim-9, wherein 90% of the particle by volume are present in the range of 20 μm to 80 μm and a suitable carrier.

12. A pharmaceutical composition comprising an iron containing phosphate adsorbents according to claim-10, having a BET active surface area less than 15 m²/gm and a suitable carrier.
